(19) United States
(12) Patent Application Publication
(10) Pub. No.: US 2011/0046698 A1
(43) Pub. Date: Feb. 24, 2011

(54) RECOVERY OF A WIRELESS COMMUNICATION SESSION WITH AN IMPLANTABLE MEDICAL DEVICE

(75) Inventors: Gary P. Kivi, Maple Grove, MN (US); Javaid Masoud, Shoreview, MN (US); Yuying (Mae) Chao, Vadnais Heights, MN (US)

Correspondence Address: MEDTRONIC, INC., 710 MEDTRONIC PARKWAY NE, MINNEAPOLIS, MN, 55432-9924, US

(73) Assignee: Medtronic, Inc.

(21) Appl. No.: 12/565,859

(22) Filed: Sep. 24, 2009

Related U.S. Application Data
(60) Provisional application No. 61/236,276, filed on Aug. 24, 2009.

(51) Int. Cl. A61N 1/08

(52) U.S. Cl. 607/60

(57) ABSTRACT

Techniques are described for recovery of an inadvertently lost communication session between an implantable medical device (IMD) and another device. For example, the IMD or other device may detect loss of the established communication session, attempt to reestablish the communication session on a same channel as the established communication session that was lost, and attempt to reestablish the communication session on an unspecified channel using a telemetry wakeup feature upon the failure to reestablish the communication session on the same channel.

### TECHNICAL FIELD

The disclosure relates generally to implantable medical devices and, in particular, to recovery of a wireless communication session between an implantable medical device and another device.

### BACKGROUND

A wide variety of implantable medical devices (IMDs) that deliver a therapy to or monitor a physiologic or biological condition of a patient, or both, have been clinically implanted or proposed for clinical implantation in patients. An IMD may deliver therapy to or monitor a physiological or biological condition with respect to a variety of organs, nerves, muscles or tissues of the patients, such as the heart, brain, stomach, spinal cord, pelvic floor, or the like. The therapy provided by the IMD may include electrical stimulation therapy, drug delivery therapy or the like.

The IMD may exchange communications with another device. The IMD may exchange communications with an external device, such as a programming device or a monitoring device (e.g., either attached to the patient or otherwise located near the patient). Alternatively, or additionally, the IMD may communicate with another implantable device, e.g., another device that forms part of an intra-body communications network. The information exchanged may be information related to a condition of the patient, such as physiological signals measured by one or more sensors, or information related to a therapy delivered to the patient. This information may be previously stored or real-time information. The IMD may also receive information from the programming device, such as configuration information that may be used to configure a therapy to be provided to the patient.

The IMD and the other device may exchange information using radio frequency (RF) communications. For example, the IMD and the other device may communicate in the 402-405 megahertz (MHz) frequency band in accordance with the Medical Implant Communications Service (MICS) band regulations. As another example, the IMD and the other device may communicate over the 401-402 MHz or 405-406 MHz frequency bands in accordance with the Medical External Data Service (MEDS) band regulations. In either case, the IMD and/or the other device with which the IMD communicates may establish a communication session between the devices over a selected communication channel. The communication session between the devices may be inadvertently lost due to any of a variety of reasons, such as environment noise.

### SUMMARY

This disclosure describes techniques for reestablishing a lost communication session between an IMD and at least one other device, a process sometimes referred to as channel recovery. In response to detecting loss of the communication session, for example, the IMD and the other device may perform channel recovery in two stages: (1) a same channel recovery and (2) an unspecified channel recovery. In same channel recovery, the IMD and the other device attempt to reestablish the communication session on the same channel on which the previously established communication session was lost using native mode packets, e.g., open request and response packets.

In unspecified channel recovery, which may begin immediately after or within a short time period after the time allotted for same channel recovery, recovery is attempted utilizing the wakeup feature of telemetry modules of the devices. As will be described in more detail below, one of the devices selects a channel on which to attempt to reestablish the communication session and transmits one or more wakeup packets followed by one or more open request packets on the selected channel. The other one of the devices listens for a wakeup packet on the plurality of the channels of the frequency band and, when a wakeup packet is received, listens for an open request packet. In response to receiving a valid open request packet, one or more open response packets are transmitted to reestablish the communication session.

In one example, this disclosure is directed to a device comprising an antenna and a telemetry module coupled to the antenna. The telemetry module is configured to detect loss of an established communication session with a telemetry module of a device, attempt to reestablish the communication session on a same channel as the established communication session that was lost, and attempt to reestablish the communication session on any channel of a plurality of available channels using a wakeup feature of the telemetry module upon the failure to reestablish the communication session on the same channel.

In another example, this disclosure is directed to a method comprising detecting loss of an established communication session with a telemetry module of a device, attempting to reestablish the communication session on a same channel as the established communication session that was lost, and attempting to reestablish the communication session on any channel of a plurality of available channels using a wakeup feature of the telemetry module upon the failure to reestablish the communication session on the same channel.

In a further example, this disclosure is directed to a device comprising means for detecting loss of an established communication session with a telemetry module of a device, means for attempting to reestablish the communication session on a same channel as the established communication session that was lost, and means for attempting to reestablish the communication session on any channel of a plurality of available channels using a wakeup feature of the telemetry module upon the failure to reestablish the communication session on the same channel.

This summary is intended to provide an overview of the subject matter described in this disclosure. It is not intended to provide an exclusive or exhaustive explanation of the techniques as described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF THE DRAWINGS

FIG. 1 is a conceptual diagram illustrating an example medical system in which at least one device uses the communication session recovery techniques described in this disclosure.

FIG. 2 is a block diagram illustrating an example IMD and external device in further detail.

FIG. 3 is a block diagram illustrating telemetry modules of the IMD and external device of FIG. 2 in further detail.

FIG. 4 is a flow diagram illustrating example operation of a telemetry module reestablishing a communication session.

FIG. 5 is a flow diagram illustrating example operation of a telemetry module reestablishing a communication session.

### DETAILED DESCRIPTION

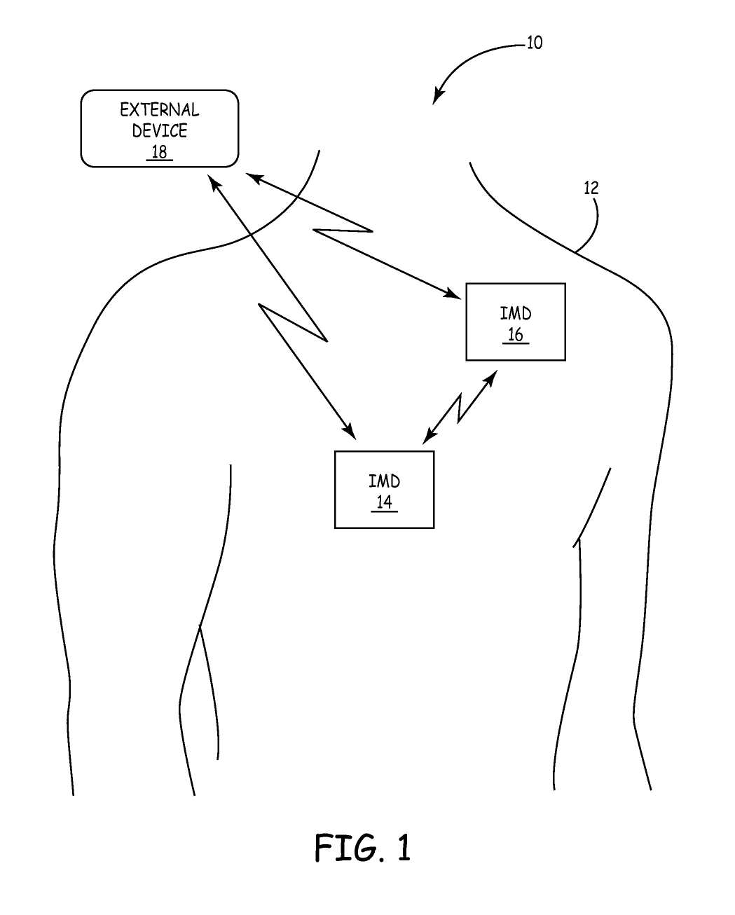

FIG. 1 is a conceptual diagram illustrating an example medical system 10 in which at least one device uses the communication session recovery techniques described in this disclosure. The medical devices of medical system 10 may include one or more medical devices that may be used to provide therapy to and/or sense one or more physiological signals of a patient 12. The medical devices of medical system 10 may also include devices that interact with IMDs to program the IMDs and/or retrieve date from the IMDs, such as programming devices and/or monitoring devices. In the example illustrated in FIG. 1, medical system 10 includes an IMD 14, IMD 16, and external device 18. Medical system 10 may, however, include more or fewer medical devices that may or may not be implanted within patient 12.

IMD 14 may be any of a variety of medical devices that provide therapy to patient 12, sense physiological or biological conditions of patient 12 or a combination thereof. In some instances, IMD 14 may be a device that provides electrical stimulation therapy in the form of cardiac rhythm management therapy to a heart of patient 12. In such a case, IMD 14 may include one or more implantable leads (not shown) with one or more electrodes that extend from IMD 14 for delivering therapy to and/or sensing physiological signals of a heart of patient 12. The leads may be implanted within one or more atria or ventricles of the heart of patient 12 or a combination thereof. In other words, IMD 14 may be used for single chamber or multi-chamber cardiac rhythm management therapy. The cardiac rhythm management therapy delivered by IMD 14 may include, for example, pacing, cardioversion, defibrillation and/or cardiac resynchronization therapy (CRT). In other instances, IMD 14 may be a device that provides electrical stimulation to a tissue site of patient 12 proximate a muscle, organ or nerve, such as a tissue proximate a vagus nerve, spinal cord, brain, stomach, pelvic floor or the like to treat various conditions, including movement and affective disorders such as chronic pain, Parkinson's disease, tremor and dystonia, urinary storage and voiding dysfunction, digestion dysfunction, sexual dysfunction or the like.

In other instances, IMD 14 may be a device that delivers a drug or therapeutic agent to patient 12 via an implantable catheter (not shown). IMD 14 may, for example, be implanted within a subcutaneous pocket in an abdomen of patient 12 and the catheter may extend from IMD 14 into the stomach, pelvic floor, brain, intrathecal space of the spine of patient 12 or other location depending on the application. IMD 14 may deliver the drug or therapeutic agent via the catheter to reduce or eliminate the condition of the patient and/or one or more symptoms of the condition of the patient. For example, IMD 14 may deliver morphine or ziconotide to reduce or eliminate pain, baclofen to reduce or eliminate spasticity, chemotherapy to treat cancer, or any other drug or therapeutic agent to treat any other condition and/or symptom of a condition.

Like IMD 14, IMD 16 may also be any of a variety of implantable medical devices that sense a physiological or biological condition of and/or deliver therapy to patient 12. As one example, IMD 16 may be a wireless (or leadless) sensor implanted within patient 12 to sense one or more physiological signals of patient 12. IMD 16 may be implanted at targeted monitoring sites and transmit the sensed signals, thus avoiding limitations associated with lead-based sensors. In some instances, IMD 16 uses the sensed physiological signals to monitor a condition of patient 12 or provide therapy to patient 12 as a function of the sensed physiological signals. Alternatively, or additionally, IMD 16 transmits the sensed physiological signals to another device, such as IMD 14 or external device 18, which may in turn monitor the condition of patient 12 or provide therapy to patient 12 as a function of the sensed physiological signals. IMD 16 may sense, sample, and process one or more physiological signals such as heart activity, muscle activity, brain electrical activity, intravascular pressure, blood pressure, blood flow, acceleration, displacement, motion, respiration, or blood/tissue chemistry, such as oxygen saturation, carbon dioxide, pH, protein levels, enzyme levels or other parameter.

Although IMD 16 is described with reference to FIG. 1 as being a wireless sensor, IMD 16 may be any of a variety of other medical devices that deliver therapy, sense physiological signals or both. For example, IMD 16 may be a leadless pacer (sometimes referred to as a wireless pacer). Other examples of medical devices that IMD 16 could be include therapy delivery devices, such as electrical stimulation devices that deliver electrical stimulation to a heart, brain, spinal cord, stomach, pelvic floor or other location within or on patient 12, or drug pumps or infusion pumps that delivers a drug, therapeutic agent, saline solution, or other liquid to locations within patient 12.

External device 18 may be a programming device or monitoring device that allows a user, e.g., physician, clinician or technician, to configure a therapy delivered by IMDs 14 and/or 16 or to retrieve data sensed by IMDs 14 and/or 16. External device 18 may include a user interface that receives input from the user and/or displays data to the user, thus allowing the user to program the therapy delivered by IMDs 14 and/or 16 or display data retrieved from IMDs 14 and/or 16. External device 18 may be a dedicated hardware device with dedicated software for programming or otherwise communicating with IMDs 14 and/or 16. Alternatively, external device 18 may be an off-the-shelf computing device running an application that enables external device 18 to program or otherwise communicate with IMDs 14 and/or 16. In some examples, external device 18 may be a handheld computing device that may be attached to or otherwise carried by patient 12. Alternatively, external device 18 may be a computer workstation, such as a CareLink® monitor, available from Medtronic, Inc. of Minneapolis, Minn.

IMD 14, IMD 16 and external device 18 may communicate with one another by any of a number of wireless communication techniques. In some instances, IMD 14, IMD 16 and external device 18 may be communicatively coupled with each other as well as other medical devices (not shown) to form a local area network, sometimes referred to as a body area network (BAN) or personal area network (PAN). Each device may therefore be enabled to communicate wirelessly along multiple pathways with each of the other networked devices. As such, IMD 14, IMD 16 and external device 18 may represent a distributed system of devices that cooperate to monitor a condition of and/or provide therapy to patient 12.

Example wireless communication techniques include RF telemetry, but other techniques are also contemplated, including inductive telemetry, magnetic telemetry, acoustic telemetry, or the like. In one instance, IMD 14, IMD 16 and/or external device 18 may communicate in accordance with the Medical Implant Communications Service (MICS) band regulation and/or the Medical External Data Service (MEDS) frequency band regulation. The MICS band regulation defines communication requirements for the 402-405 MHz frequency band. In accordance with the MICS band regulations, the frequency band is divided into ten channels with each channel corresponding to a 300 kilohertz (kHz) sub-band. The MEDS band regulation defines a split channel band with a portion of the MEDS band occupying the 401-402 MHz frequency band and a portion of the MEDS band occupying the 405-406 MHz frequency band. The MEDS band is divided into 20 channels with each channel corresponding to a 100 kHz sub-band, with the first ten channels being located in the 401-402 MHz frequency band and the second ten channels being located in the 405-406 MHz frequency band. The devices of medical system 10 may, however, communicate using any frequency band regulation in addition to or instead of the MICS and MEDS band regulations.

To establish a communication session, one of the devices of medical system 10 may select one of the channels of the frequency band to transmit on. The device may, for example, perform a clear-channel assessment (CCA) to select the one of the channels of the frequency band with a lowest ambient power level (e.g., the least-noisy or least-interfered with channel). Typically, external device 18 performs the CCA process. However, in other instances, one or both of IMD 14 and 16 may be configured to perform the CCA process, particularly in the context of intra-body wireless communication between IMD 14 and IMD 16. Performing CCA increases the likelihood that the external device 18 selects an unused channel, thus decreasing the likelihood of interference from multiple communication sessions attempting to use the same channel. In other instances, none of the devices may perform CCA. Instead, the devices may begin to transmit over any channel without regard to noise or use of the channel.

Once the external device 18 selects the channel to transmit on (e.g., either by CCA or randomly), external device 18 establishes a communication session with one of IMDs 14 or 16 over the selected channel by exchanging one or more packets. For purposes of explanation, external device 18 will be described as establishing a communication session with IMD 14. However, external device 18 may establish a communication session with IMD 16 in addition to or instead of IMD 14. Additionally, a communication session may be established between IMD 14 and IMD 16 in a similar manner.

Once the communication session is established, external device 18 and IMD 14 may transmit communications to and receive communications from one another. External device 18 may, for example, transmit information to IMD 14, such as configuration information that may be used to configure a therapy to be provided to patient 12. IMD 14 may transmit information to the external device 18, including information related to a condition of patient 12, information related to a therapy delivered to patient 12, or information related to a status of one or more components of IMD 14 or components coupled to IMD (e.g., leads).

The communication session between external device 18 and IMD 14 may be inadvertently lost. For example, the communication session may be lost due to any of a variety of environmental noises, which may be transient or persistent. The noise may result in external device 18 or IMD 14 receiving unintelligible information or no information at all. As another example, the communication session between IMD 14 and external device 18 may be lost if a received signal strength falls below a threshold. This may occur when the distance between IMD 14 and external device 18 becomes too far or an object obstructs the communication path between IMD 14 and external device 18.

Upon the communication session being inadvertently lost, the devices may perform channel recovery in accordance with the techniques of this disclosure in an effort to reestablish an active communication session. For example, IMD 14 and external device 18 may detect loss of the established communication session, attempt to reestablish the communication session on a same channel as the established communication session that was lost, and attempt to reestablish the communication session on an unspecified channel using a telemetry wakeup feature upon the failure to reestablish the communication session on the same channel.

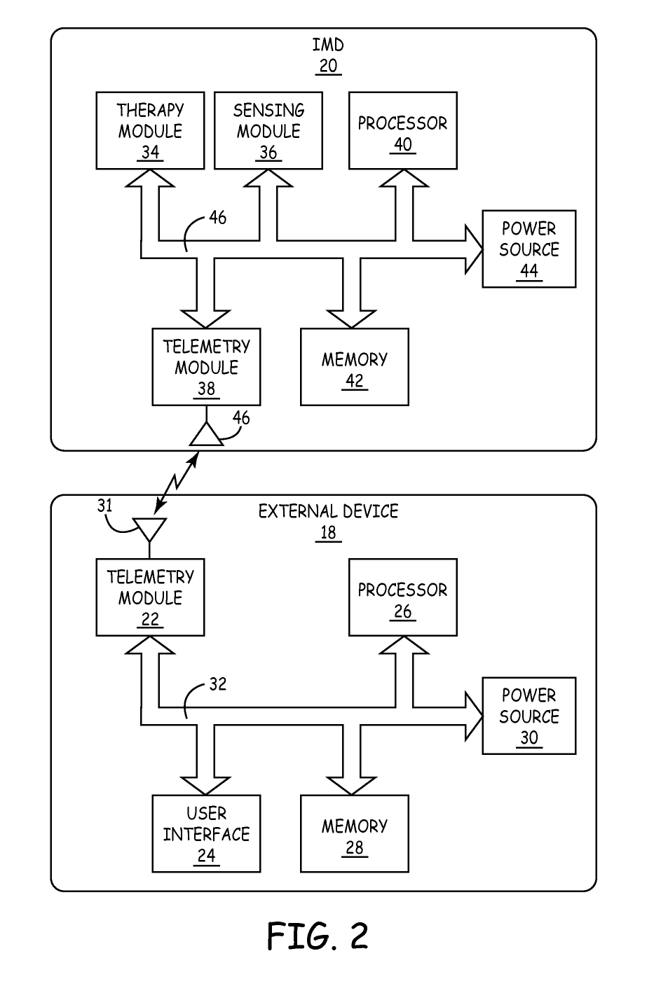

FIG. 2 is a block diagram illustrating an example IMD 20 and external device 18 in further detail. IMD 20 may correspond to IMD 14 or IMD 16 of FIG. 1. External device 18 may correspond to a programming device, a monitoring device or other external device located on or in the vicinity of patient 12. As illustrated in the example of FIG. 2, external device 18 includes a telemetry module 22, user interface 24, processor 26, memory 28 and power source 30, all of which are interconnected by a data bus 32. IMD 20 includes a therapy module 34, sensing module 36, telemetry module 38, processor 40, memory 42 and power source 44, all of which are interconnected by a data bus 46.

Power source 44 of IMD 20 may include a rechargeable or non-rechargeable battery. A non-rechargeable battery may be selected to last for several years, while a rechargeable battery may be charged from an external charging device on a daily or weekly basis. In either case, and especially in the case of the non-rechargeable battery, the amount of power of the battery is limited.

IMD 20 may sense one or more physiological signals or conditions of patient 12. In some instances, IMD 20 may not provide therapy to patient 12, but only provide monitoring of patient 12 as in the case of an implantable loop recorder. In such cases, IMD 20 may not include therapy module 34. Sensing module 36 is configured to monitor one or more physiological signals using one or more sensors connected to sensing module 36. In one example, sensing module 36 is configured to monitor signals sensed by one or more of electrodes on leads extending from IMD 20. In another example, sensing module 36 may be configured to monitor signals sensed by a sensor within or on IMD 20. In a further example, sensing module 36 may be configured to receive signals sensed by one or more wireless or lead-less sensors and transmitted wirelessly to IMD 20. The one or more sensors may sense physiological signals such as heart activity (e.g., electrocardiogram (ECG) signals), muscle activity (e.g., electromyography (EMG) signals), brain electrical activity (e.g., electroencephalography (EEG) signals), heart rate, intravascular pressure, blood pressure, blood flow, acceleration, displacement, motion, respiration, or blood/tissue chemistry such as oxygen saturation, carbon dioxide, pH, protein levels, enzyme levels or other parameter.

Sensing module 36 may store the sensed signals in memory 42. In some instances, sensing module 36 may store the sensed signals in raw form. In other instances, sensing module 36 may process the sensed signals and store the processed signals in memory 42. For example, sensing module 36 may amplify and filter the sensed signal and store the filtered signal in memory 42. The signals stored by sensing module 36 may, in some cases, be retrieved and further processed by processor 40.

IMD 20 may also provide therapy, such as electrical stimulation therapy or drug delivery therapy, to patient 12 in accordance with parameters of one or more selected therapy programs. In particular, processor 36 controls therapy module 34 to deliver therapy to patient 12 according to one or more therapy programs, which may be received from external device 18 and stored in memory 42. In the case of electrical stimulation therapy, therapy module 34 may include a stimulation generator that generates and delivers electrical stimulation therapy, e.g., in the form of pulses or shocks. Processor 40 may control the stimulation generator to deliver electrical stimulation pulses with amplitudes, pulse widths, frequency, and/or electrode polarities specified by the one or more therapy programs. In the case of drug delivery therapy, therapy module 34 may include a pump that delivers a drug or therapeutic agent to patient 12. Processor 40 may control the pump to deliver the drug or therapeutic agent with the dosage and frequency (or rate) specified by the one or more therapy programs.

Processor 40 may include any one or more of a microprocessor, a controller, a digital signal processor (DSP), an application specific integrated circuit (ASIC), a field-programmable gate array (FPGA), or equivalent discrete or integrated logic circuitry. In some examples, processor 40 may include multiple components, such as any combination of one or more microprocessors, one or more controllers, one or more DSPs, one or more ASICs, or one or more FPGAs, as well as other discrete or integrated logic circuitry. The functions attributed to processor 40 herein may be embodied as software, firmware, hardware or any combination thereof.

Memory 42 includes computer-readable instructions that, when executed by processor 40, cause IMD 20 and processor 40 to perform various functions attributed to IMD 20 and processor 40 herein. Memory 42 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, magnetoresistive random access memory (MRAM), static random access memory (SRAM) or any other digital media.

Processor 40 controls telemetry module 38 to transmit communications to and/or receive communications from another medical device, such as external device 18, via an established communication session. Telemetry module 38 may also transmit communications to and/or receive communications from other external and/or implanted medical devices. Processor 40 may provide the data to be transmitted to external device 18 and the control signals for telemetry circuitry within telemetry module 38, e.g., via data bus 46. Telemetry module 38 transmits the data to external device 18 in accordance with the control signals from processor 40. Telemetry module 38 may provide data received from external device 18 to processor 40. Processor 40 may analyze the received data, store the received data within memory 42 and configure components of IMD 20 in accordance with the received data.

Telemetry module 38 includes any suitable hardware, firmware, software or any combination thereof for communicating with another device, such as external device 18. For example, telemetry module 38 may include appropriate modulation, demodulation, frequency conversion, filtering, and amplifier components for transmission and reception of data, including radio frequency (RF) components and antenna 46, as applicable.

A user may interact with external device 18 to program IMD 20 to provide therapy in accordance with a selected therapy program and/or view data retrieved from IMD 20. The user may, for example, interact with external device 18 via user interface 24 to select therapy programs (e.g., sets of stimulation parameters), generate new therapy programs and/or modify therapy programs through individual or global adjustments. User interface 24 may include, for example, a keypad and a display, which may be, for example, a cathode ray tube (CRT) display, a liquid crystal display (LCD) or light emitting diode (LED) display. The keypad may take the form of an alphanumeric keypad or a reduced set of keys associated with particular functions. External device 18 can additionally or alternatively include a peripheral pointing device, such as a mouse, via which a user may interact with the user interface. In some embodiments, the display of external device 18 may include a touch screen display, and a user may interact with external device 18 via the display.

Processor 26 controls telemetry module 22 to transmit the parameters of the one or more selected therapy programs, which may be stored within memory 28 or directly input by the user via user interface 24, to telemetry module 38 of IMD 20. Telemetry module 22, under the control of processor 26, may also receive data from telemetry module 38 of IMD 20, which may include sensed physiological parameters, diagnosis generated based on the sensed physiological parameters, a log of delivered therapies, information regarding the amount of remaining battery power or the like. Processor 26 may store the retrieved data in memory 28 for later processing or transmission to another device, e.g., a remote server.

Telemetry module 22 communicates wirelessly with IMD 20 and, more specifically, with telemetry module 38 of IMD 20. Telemetry module 22, like telemetry module 38 of IMD 20, may include any suitable hardware, firmware, software or any combination thereof for communicating with IMD 20. For example, telemetry module 22 may include appropriate modulation, demodulation, frequency conversion, filtering, and amplifier components for transmission and reception of data, including radio frequency (RF) components and antenna 31, as applicable. In some instances, telemetry module 22 may include two or more sets of RF components, e.g., one for communication with IMD 20 and one for communication with another computing device (e.g., remote server).

Telemetry modules 22 and 38 may establish a communication session in accordance with the requirements of the frequency band regulation over which the devices are communicating and/or in accordance with one or more wireless communication protocols via which the devices are communicating. In one example, one of telemetry modules 22 or 38 selects one of the channels of the frequency band to transmit on, e.g., via a CCA. Although described below as performing CCA to select a channel, telemetry module 22 may, in some instances, begin transmitting data over a channel without performing CCA. However, performing CCA increases the likelihood that the external device 18 selects an unused channel, thus decreasing the likelihood of interference from multiple communication sessions attempting to use the same channel. Such schemes may be particularly useful in an environment in which a number of medical devices are communicating using a limited number of channels, e.g., in a hospital, nursing home, doctor's office, or the like.

Once external device 18 selects the channel to transmit on, a communication session is established between external device 18 and IMD 20 over the selected channel. To establish the communication session, external device 18 and IMD 20 exchange communications to configure the communication session. In one example, external device 18 may transmit one or more packets indicating the desire to establish (or open) a communication session on the selected channel. External device 18 may generate and transmit the one or more packets in a native communication mode, which is described in more detail below. The packets sent from the initiating device (external device 18 in this example) indicating the desire to establish a communication session will be referred to in this disclosure as “open request packets.” In response to receiving one of the open request packets, IMD 20 may transmit one or more packets indicating its desire to establish the communication session with external device 18. The packets sent from the responding device (IMD 20 in this example) in response to the open request packets are also sent in the native communication mode and are referred to herein as “open response packets.” Upon receiving an open response packet from IMD 20, the communication session is established on the selected channel.

In some instances, telemetry module 38 of IMD 20 may be in a low-power state (also known as a low current state or a sleep state) prior to the desire to communicate. Such a low power state may reduce power consumption by telemetry module 38 when no communication session is active. During the low-power state, telemetry module 38 of IMD 20 may periodically monitor one or more of the channels for a wakeup packet, a process sometimes referred to as “sniffing.” Telemetry module 38 of IMD 20 may, for example, sniff the one or more channels for a wakeup packet every two seconds. The sniff interval may be set to be a longer or shorter period of time than two seconds and may be on the order of milliseconds, minutes, hours or the like.

In this case, telemetry module 22 of external device 18 may transmit wakeup packets to IMD 20 before sending the one or more open request packets. Telemetry module 22 of external device 18 may transmit wakeup packets for a duration of time that is at least slightly longer than the sniff interval of telemetry module 38 of IMD 20. For example, telemetry module 22 of external device 18 may transmit at least sixty-five wakeup packets with each wakeup packet being about thirty-one milliseconds. As described further below, the wakeup packets may be generated and transmitted in a wakeup communication mode that is different than the native communication mode. In response to receiving a wakeup packet, telemetry module 38 may transition from the low power state to a high power state and begin to listen for an open request packet. Telemetry module 38 may begin to listen for the open request packet on the channel on which the wakeup packet was received or on a channel specified within the wakeup packet.

Once the communication session is established, telemetry module 22 of external device 18 and telemetry module 38 of IMD 20 may exchange data with one another. As described above, external device 18 may transmit configuration information to IMD 20 that may be used to configure a therapy to be provided to patient 12, e.g., parameters of one or more selected therapy programs. IMD 20 may transmit sensed physiological parameters, diagnosis generated based on the sensed physiological parameters, a log of delivered therapies, information regarding the amount of remaining battery power or other status indicator, or other data to external device 18.

The communication session established between external device 18 and IMD 20 may be inadvertently lost, thus rendering external device 18 and IMD 20 incapable of communicating with one another. The communication session may be lost due to any of a variety of reasons, including transient or persistent environmental noise, one or more objects located between external device 18 and IMD 20 that block the communication path, too large a distance between external device 18 and IMD 20, or the like. Telemetry module 22 of external device 18 and telemetry module 38 of IMD 20 may be configured to detect loss of the communication session. Telemetry modules 22 and 38 may, for example, detect loss of the communication session when a signal strength of an incoming signal falls below a threshold level, when no intelligible communication is received for a threshold period of time, e.g., a particular number of consecutive frames, when a valid communication is received from an unexpected device, or the like.

In response to detecting loss of the communication session, external device 18 and IMD 20 perform channel recovery in an effort to reestablish an active communication session. The channel recovery may, in some instances, involve two stages: (1) a same channel recovery and (2) an unspecified channel recovery. For example, external device 18 and IMD 20 may try to reestablish the communication session using same channel recovery for a particular period of time and, if unsuccessful, attempt to reestablish the communication session using unspecified channel recovery. In same channel recovery, external device 18 and IMD 20 attempt to reestablish the communication session on the same channel on which the previously established communication session was lost using open request and response packets. Same channel recovery may, for example, be successful if the environmental noise that caused the communication session to fail was transient and is no longer present, if an interfering object is removed or if the distance between external device 18 and IMD 20 is shortened.

In unspecified channel recovery, which may begin substantially immediately after or within a short time period after the time allotted for same channel recovery, recovery is attempted utilizing the wakeup features of telemetry modules 22 and 38. As will be described in more detail below, telemetry module 22 of external device 18 may select a channel on which to attempt to reestablish the communication session and transmit one or more wakeup packets followed by one or more open request packets on the selected channel. Telemetry module 38 of IMD 20 may listen for a wakeup packet on a plurality of the channels of the frequency band for one or more listening periods, e.g., two or more asynchronous receptor wakeup intervals. Unlike the wakeup technique described for initially establishing the communication session, telemetry module 38 of IMD 20 may not be in a low-power state. Thus, the wakeup sniffing occurs in the high power state to provide a faster technique for IMD 20 to identify the channel selected by telemetry module 22 of external device 12.

When a wakeup packet is received, telemetry module 38 of IMD 20 listens for an open request packet on the channel on which the wakeup packet was detected or a channel specified within the wakeup packet. Telemetry module 38 of IMD 20 transmits one or more open response packets in response to receiving a valid open request packet. The communication session is reestablished using unspecified channel recovery when telemetry module 22 of external device 18 receives a valid open response packet from IMD 20. In this manner, IMD 20 and external device 18 may utilize a wakeup feature of the telemetry modules to reestablish the communication session.

In one aspect, the transmission and reception of wakeup packets and open request packets are performed using different encoding schemes, different transmission or data rates, different packet sizes, different packet structures or the like. In one example, the wakeup communication mode uses Manchester encoding, a data rate of 6.4 Kbps, a packet size of 25 bytes and the packet includes content bytes and MAC bytes while the native communication mode uses DQPSK or DBPSK encoding, a data rate of greater than 48 Kbps, a packet size of greater than 47 bytes and a packet structure that includes control, content, reed Solomon and MAC bytes. As such, a less complicated communication mode (i.e., the wakeup communication mode) is used to transition IMD 20 from a low power state to a high power state and the more complex communication mode (i.e., the native communication mode) is used to send and/or receive data from IMD 20.

Although in the example described above, one or more components of external device 18, e.g., telemetry module 22 or processor 26, initiates the reestablishment of the communication session by sending the open request packets and/or wakeup packets, the initiation of the reestablishment process may be performed by one or more components of IMD 20. In other words, telemetry module 38 and/or processor 40 may send one or more open request packets and/or wakeup packets to external device 18. This may be the case in which two or more implanted medical devices, e.g., IMD 14 and 16 of FIG. 1, communicate using intra-body wireless communication.

Processor 26 may include one or more of a microprocessor, a controller, a DSP, an ASIC, an FPGA, or equivalent discrete or integrated logic circuitry. In some examples, processor 26 may include multiple components, such as any combination of one or more microprocessors, one or more controllers, one or more DSPs, one or more ASICs, or one or more FPGAs, as well as other discrete or integrated logic circuitry. The functions attributed to processor 26 herein may be embodied as software, firmware, hardware or any combination thereof.

Memory 28 includes computer-readable instructions that, when executed by processor 26, cause external device 18 and processor 26 to perform various functions attributed to external device 18 and processor 26 herein. Memory 28 may include any volatile, non-volatile, magnetic, optical, or electrical media, such as a RAM, ROM, NVRAM, EEPROM, flash memory, MRAM, SRAM, or any other digital media.

Power source 30 of external device 18 delivers operating power to the components of external device 18. Power source 30 may include a battery and a power generation circuit to produce the operating power for the components of external device 18. In some examples, the battery may be rechargeable (e.g., nickel cadmium or lithium ion batteries) to allow extended operation. Recharging may be accomplished by electrically coupling power source 30 to a cradle or plug that is connected to an alternating current (AC) outlet. In addition or alternatively, recharging may be accomplished through proximal inductive interaction between an external charger and an inductive charging coil within external device 18. In other embodiments, non-rechargeable batteries (e.g., non-rechargeable lithium based batteries such as lithium iodide) may be used. In addition, external device 18 may be directly coupled to an AC outlet to power external device 18.

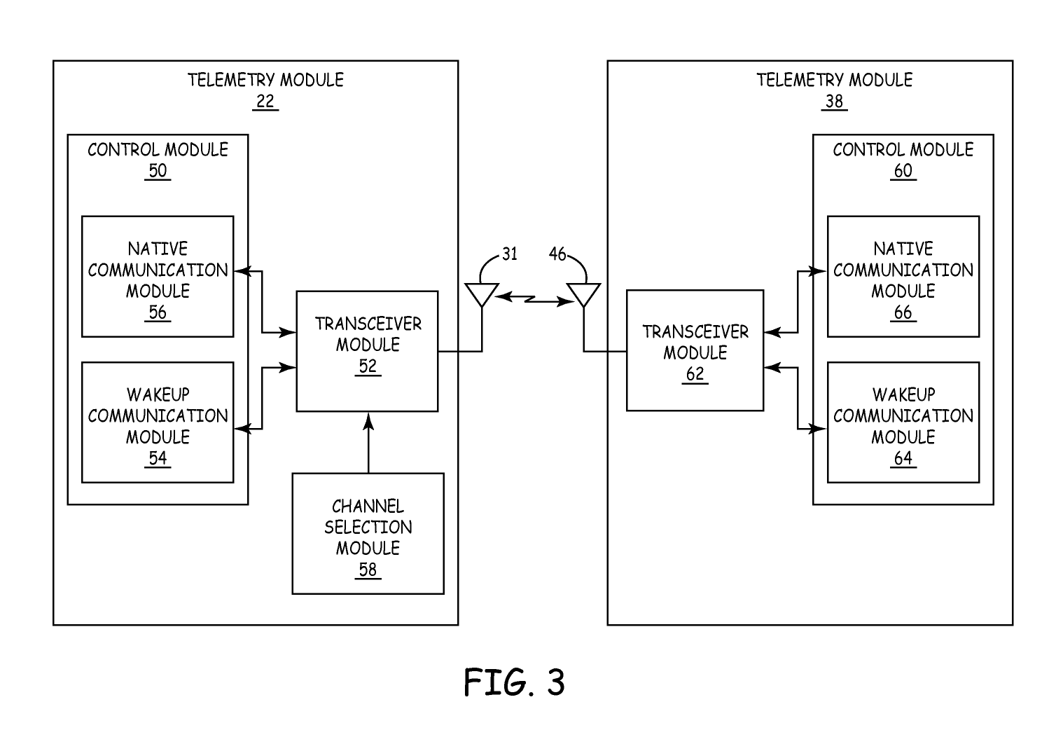

FIG. 3 is a block diagram illustrating telemetry modules 22 and 38 in further detail. Telemetry module 22 includes a control module 50, a transceiver module 52, and a channel selection module 58. Control module 50 further includes a wakeup communication module 54 and a native communication module 56, which generate and transmit communications in a wakeup communication mode and native communication mode respectively. Transceiver module 52 is coupled to an antenna 31. Telemetry module 38 includes a control module 60 and a transceiver module 62. Control module 60 further includes a wakeup communication module 64 and a native communication module 66, which generate and transmit communications in a wakeup communication mode and native communication mode respectively. Transceiver module 62 is coupled to an antenna 46.

When external device 18 desires to establish a communication session with IMD 20, e.g., in response to an input from a user of external device 18 or in response to receiving a signal from IMD 20, channel selection module 58 of telemetry module 22 may select a channel to transmit on. In one instance, channel selection module 58 may select the channel to transmit on via CCA. During CCA, channel selection module 58 monitors at least a portion of the channels of the frequency band(s) and selects one of the channels that meets a particular condition. In one example, channel selection module 58 monitors all of the channels and selects the channel with the lowest ambient power level (the least-noisy or least-interfered with channel) as the channel to transmit on. In another example, channel selection module 58 assesses at least a portion of the channels of the frequency band, and in some instances all of the channels of the frequency band, as a function of the ambient power on the respective channels and the ambient power on at least one other channel, e.g., the two immediately adjacent channels. Such a technique is described in copending U.S. patent application Ser. No. 12/414,946 to Corndorf, which is incorporated herein by reference in its entirety. In another example, channel selection module 58 may monitor the channels of the frequency band and select a first channel having an ambient power level below a minimum power level threshold. In other instances, channel selection module 58 may select a channel to transmit on without monitoring the channels.

Once external device 18 selects the channel to transmit on, a communication session is established between external device 18 and IMD 20 over the selected channel. In one example, wakeup communication module 54 generates one or more wakeup packets and provides the wakeup packets to transceiver module 52 for transmission on the selected channel. As described above, the wakeup packets indicate to telemetry module 38 of IMD 20 that it should wakeup from a low-power state and monitor for native mode packets. The wakeup packets are communication in the wakeup communication mode which may be slightly less complicated than the native communication mode. The wakeup communication mode may, for example, have a less complicated encoding schemes, slower transmission or data rate, smaller packet sizes, and/or simpler packet structures than the native communication mode.

Following transmission of the one or more wakeup packets, native communication module 56 generates one or more open request packets (or other native mode packets) and provides the open request packets to transceiver module 52 for transmission on the selected channel. As described above, the open request packets indicate the desire of the initiating device to establish (or open) a communication session on the selected channel. The open request packets or other native mode packets are communication in the native communication mode which may be more complex than the wakeup communication mode. The native communication mode may, for example, have a more complex encoding scheme (e.g., DQPSK or DBPSK vs. Manchester), faster transmission or data rate (e.g., >47 Kbps vs. 6.4 Kbps), larger packet sizes (e.g., >47 bytes vs. 25 bytes), and/or more complex packet structures than the wakeup communication mode.

After transmission of the open request packets, native communication module 56 monitors for an open response packet from IMD 20. If an open response packet is received from IMD 20, the communication session is established on the selected channel. If no open response packet is received from IMD 20, telemetry module 22 may reattempt to establish the communication session by either sending additional open response packets and/or additional wakeup packets or determine that no communication session can be established at this time.

Prior to the initial establishment of a communication session, telemetry module 38 of IMD 20 may be in a low power state in which one or more components of telemetry module 38 are powered down or operating in a low current state. During the low power state, wakeup communication module 64 may periodically monitor or sniff one or more of the channels for a wakeup packet. For example, wakeup communication module 64 may sniff the one or more channels for a wakeup packet every two seconds. If no wakeup communication is received during a first sniff interval, wakeup communication module 64 waits for a period of time to expire (e.g., two seconds) before listening for a second sniff interval. In some instances, wakeup communication module 64 may sniff on a plurality of channels concurrently. One example technique for sniffing on a plurality channels concurrently is described in detail in copending U.S. patent application Ser. No. 12/364,432 to Bradley et al, which is incorporated herein in its entirety.

In some aspects, wakeup communication module 64 may perform the sniff operations in a plurality of phases or stages to monitor for a wakeup signal. Each of the phases or stages may consume different amounts of power, with the first stage consuming the least amount of power and the last stage consuming the most amount of power. Moreover, each of the previous stages may be required to be satisfied before the subsequent stage is invoked. If each of the phases or stages of the sniff are met (i.e., the signal satisfies particular criteria of the stage), a valid wakeup signal is detected. If any of the stages is not met, the sniff is aborted.

In one example, a first detection phase may be a power level comparator. The first detection phase will abort when the received signal strength indication (RSSI) average for a given signal, or other power level indicator of the signal, is not above a threshold level. This technique may block most ambient channel noise from passing through the first detection phase. On passing the first detection phase, the received signal will be subjected to a second detection phase that may monitor an Integrated Frequency Deviation. The second detection phase may, for example, estimate the frequency deviation of the incoming signal and compare this with both high and low thresholds. Continuous Wave (CW) signals will “abort low” based on near-zero frequency deviation. Ambient noise signals which passed through the first detection phase, as well as other high bandwidth signals, will abort high. On passing through the second detection phase, the received signal will be subjected to a third detection phase. The third detection phase demodulates the signal and counts the number of Manchester-encoding errors. The third detection phase will abort when the number of Manchester errors exceeds a threshold. When the number of errors does not exceed the threshold, a valid wakeup signal is detected.

Other multi-phased or multi-staged wakeup detection techniques may be used, however. For example, each of the phases may be initiated concurrently and may all be aborted in response to the received signal not meeting the requirements of any one of the phases. Other example phased wakeup detection techniques are described in copending U.S. patent application Ser. No. 12/242,789 to LeReverend et al. and copending U.S. patent application Ser. No. 12/242,782 to LeReverend et al., both of which are incorporated herein in their entirety. In any case, performing the sniff operations in a plurality of stages or phases to monitor for wakeup signal enables quick abortion of the sniff on a channel when early stage requirements are not met.

In response to detecting a wakeup packet, telemetry module 38 may transition from the low power state to a high power state and begin to listen for an open request packet. For example, the one or more components that were powered down or in a low current state are powered up for operation. One such component may be native communication module 66. Native communication module 66 begins to listen for the open request packet on the channel on which the wakeup packet was received or on a channel specified within the wakeup packet. In response to receiving one of the open request packets, native communication module 66 generates one or more open response packets and provides the open response packets to transceiver module 62 for transmission.

Once the communication session is established (e.g., telemetry module 22 receives an open response packet from telemetry module 38), telemetry module 22 of external device 18 and telemetry module 38 of IMD 20 may exchange data with one another via the active communication session. The active communication session between telemetry module 22 of external device 18 and telemetry module 38 of IMD 20 may be inadvertently lost due to any of the variety of reasons discussed above. Telemetry module 22 of external device 18 and telemetry module 38 of IMD 20 may be configured to detect loss of the communication session. For example, native communication modules 46 and 56 may each include a session loss detection module (not shown) that detects loss of the communication session when a signal strength of an incoming signal falls below a threshold level, when no intelligible communication is received for a threshold period of time, e.g., a particular number of consecutive frames, when a valid communication is received from an unexpected device, or the like.

In response to detecting loss of the communication session, telemetry modules 22 and 38 perform channel recovery in an effort to reestablish an active communication session. As described above, the channel recovery may first be attempted on the same channel using native mode communications and, if unsuccessful, an attempt on an unspecified channel may be performed using a combination of wakeup mode communications and native mode communications. During the same channel recovery, telemetry module 22 and 38 attempt to reestablish the communication session on the same channel on which the previously established communication session was lost. For example, native communication module 56 of telemetry module 22 transmits one or more open request packets on the channel on which the communication session was most recently established indicating the desire to establish (or open) a communication session on that channel. Native communication module 66 of telemetry module 38 listens on the channel on which the communication session was most recently established for an open request packet and transmits one or more open response packets when a valid open request packet is detected. The communication session is reestablished using same channel recovery when native communication module 56 receives a valid open response packet from telemetry module 38. As such, the same channel recovery is performed in the native communication mode.

Telemetry module 38 of IMD 20 and telemetry module 22 of external device 18 attempt to reestablish the communication session using same channel recovery for a specified period of time. In one example, telemetry modules 22 and 38 may attempt to reestablish the communication session using same channel recovery for approximately 400 milliseconds. However, telemetry modules 22 and 38 may attempt to reestablish the communication session using same channel recovery for other periods of time longer than or shorter than 400 milliseconds. Telemetry modules 22 and 38 may each maintain a same channel recovery timer to track the amount of time that has elapsed since beginning the same channel recovery.

If the communication session is not reestablished within the period of time allotted for same channel recovery, e.g., the same channel recovery timer expires or exceeds a threshold, external device 18 and IMD 20 attempt to reestablish the communication session using unspecified channel recovery. Unspecified channel recovery may begin substantially immediately after or within a short time period after the period of time allotted for same channel recovery has expired. In the unspecified channel recovery, channel selection module 58 selects a channel on which to attempt to reestablish the communication session. Channel selection module 58 may select the channel using the CCA techniques described above. The selected channel will, in most instances, be a channel other than the channel on which the previous communication session was established (i.e., the communication session that was just lost). In some instances, however, the selected channel may be the same channel on which the previous communication session was established.

In unspecified channel recovery, wakeup communication module 54 of telemetry module 22 generates one or more wakeup packets and provides the wakeup packets to transceiver module 52 for transmission on the selected channel. As described above, the wakeup packets are transmitted in accordance with a communication mode different than the native communication mode. Following transmission of the wakeup packets, native communication module 56 of telemetry module 22 generates one or more native packets, e.g., open request packets, and provides the native packets to transceiver module 52 for transmission on the selected channel.

During unspecified channel recovery, wakeup communication module 64 of telemetry module 38 listens for a wakeup packet on a plurality of the channels of the frequency band. Wakeup communication module 64 may concurrently monitor more than one of the plurality of channels for a wakeup packet as described in more detail above. Additionally, or alternatively, wakeup communication module 64 may monitor or sniff for the wakeup packet using a plurality of phases or stages that enable early abortion of the sniff when particular conditions are met.

In some instances, wakeup communication module 64 monitors for a wakeup packet for more than one listening periods, e.g., two or more asynchronous receptor wakeup intervals. In other words, wakeup communication module 64 may monitor the channels for a wakeup packet during a first sniff interval and, if no wakeup packet is received, monitor the channels for a wakeup packet during a second, consecutive sniff interval. The second, consecutive sniff interval (second period of time) may occur shortly and, in some cases substantially immediately, after the first sniff interval (first period of time) is over. As such, wakeup communication module 64 may forego waiting the period of time before listening for the second interval, as is done during typical wakeup described above with respect to the initial establishment of the communication session.

When a wakeup packet is detected or received, native communication module 66 telemetry module 38 listens for an open request packet on the channel on which the wakeup packet was detected or a channel specified within the wakeup packet. Unlike the wakeup technique described for initially establishing the communication session, telemetry module 38 may not be in a low-power state while monitoring for wakeup packets. Thus, the wakeup sniffing occurs in the high power state to provide a faster technique for telemetry module 38 to identify the channel selected by telemetry module 22 for channel recovery. Moreover, the ability to abort the sniff early increases the quickness with which telemetry module 38 may monitor for wakeup packets.

Native communication module 66 transmits one or more open response packets in response to receiving a valid open request packet. The communication session is reestablished using unspecified channel recovery when telemetry module 22 receives a valid open response packet from telemetry module 38. In this manner, telemetry modules 22 and 38 may utilize the wakeup feature to reestablish the communication session with reduced latency.

Transceiver modules 52 and 62 transmit and receive signals, e.g., via radio frequency (RF) communication, via antennas 31 and 46, respectively. Transceiver module 52 may include any suitable hardware, firmware, software or any combination thereof for transmitting signals, including appropriate modulation, demodulation, frequency conversion, filtering, and amplifier components for transmission and reception of data.

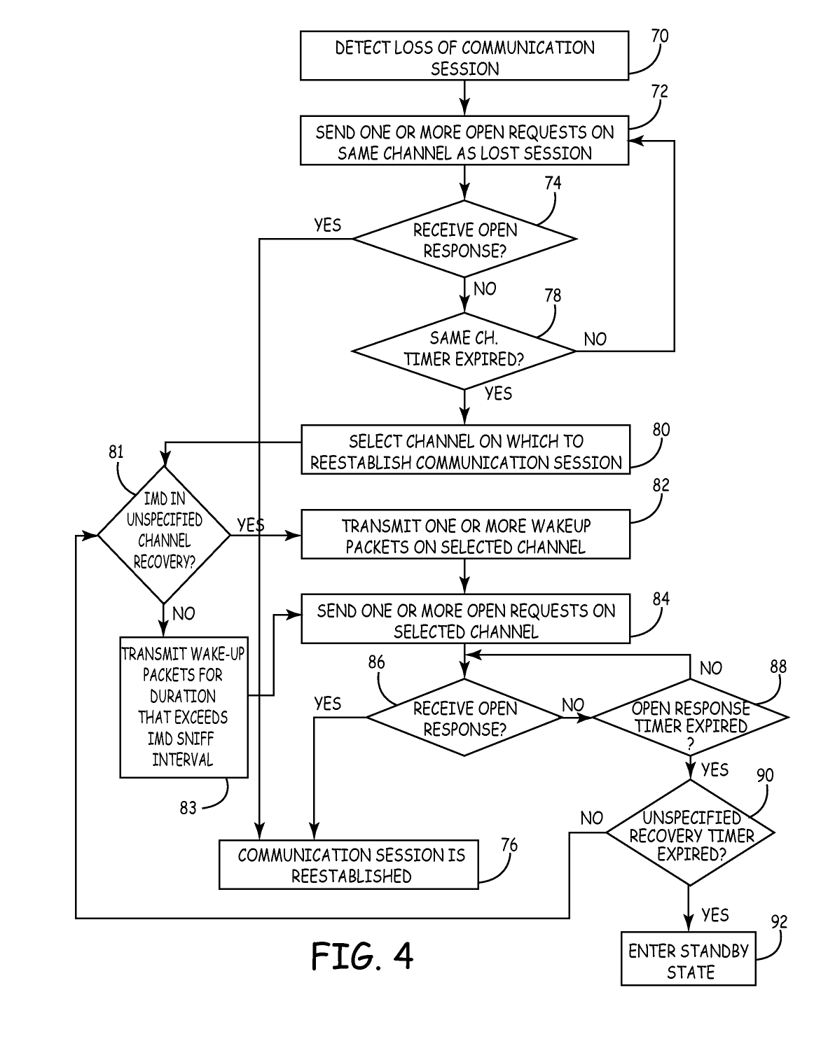

FIG. 4 is a flow diagram illustrating example operation of a telemetry module 22 of an external device 18 reestablishing a communication session with an IMD 20. Initially, telemetry module 22 detects loss of an established communication session with an IMD 20 (70). As described above, telemetry module 22 of external device 18 may detect loss of an established communication channel when no valid packet is received from IMD 20 for a particular number of consecutive frames, when a valid packet is received but from a device other than IMD 20, e.g., an unexpected IMD or another external device other than IMD 20, or the like. In response to detecting loss of the established communication session, telemetry module 22 of external device 18 sends one or more open request packets on the same channel as the established communication session that was lost (72). The open request packets are native mode packets.

Telemetry module 22 of external device 18 listens to determine whether any open response packets are received from IMD 20 (74). When an open response packet is received from IMD 20 (“YES” branch of 74), the communication channel is reestablished with IMD 20 on the same channel as lost session (76). When no open response packet is received (“NO” branch of 74), telemetry module 22 of external device 18 determines whether a same channel recovery timer has expired (78). When the same channel recovery timer has not expired (“NO” branch of 78), telemetry module 22 of external device 18 sends one or more additional open packets on the same channel as the lost communication session and listens for open responses. Alternatively, telemetry module 22 of external device 18 may not send additional open packets, but instead just listen for open responses.

When the same channel recovery timer has expired (“YES” branch of 78), telemetry module 22 of external device 18 aborts the same channel recovery and attempts to reestablish communication with IMD 20 using unspecified channel recovery. In the unspecified channel recovery, telemetry module 22 of external device 18 selects a channel on which to reestablish the communication session with IMD 20 (80). Telemetry module 22 of external device 18 determines whether telemetry module 38 of IMD 20 is still performing unspecified channel recovery (81). As described above, telemetry module 22 of external device 18 may specify the unspecified channel recovery duration to IMD 20 upon initially establishing the communication session. Telemetry module 22 may use this duration to determine whether telemetry module 38 of IMD 20 is still performing unspecified channel recovery. When telemetry module 38 of IMD 20 is still performing unspecified channel recovery (“YES” branch of 81), telemetry module 22 of external device 18 transmits one or more wakeup packets on the selected channel (82). When telemetry module 38 of IMD 20 is not performing unspecified channel recovery (“NO” branch of 81), telemetry module 22 of external device 18 transmits wakeup packets for a duration that slightly exceeds a standby state sniff interval of IMD 20 (83). In either case, external device 18 transmits one or more open request packets subsequent to the transmission of the one or more wakeup packets (84).

Telemetry module 22 of external device 18 listens to determine whether any open response packets are received from IMD 20 (86). When an open response packet is received from IMD 20 (“YES” branch of 86), the communication channel is reestablished with IMD 20 on the selected channel (76). When no open response packet is received (“NO” branch of 86), telemetry module 22 of external device 18 determines whether an open response timer has expired (88). When the open response timer has not expired (“NO” branch of 88), telemetry module 22 continues to listen for an open response packet.

When the open response timer has expired (“YES” branch of 88), telemetry module 22 of external device 18 determines whether an unspecified channel recovery timer has expired (90). When the unspecified channel recovery timer has not expired (“NO” branch of 90), telemetry module 22 of external device 18 transmits one or more additional wakeup packets, followed by one or more additional open request packets, and listens for open response packets. When the unspecified channel recovery time has expired (“YES” branch of 90), telemetry module 22 of external device 18 may enter a standby state (92). In other words, if neither the same channel recovery nor the unspecified channel recovery is successful, telemetry module 22 may enter a low power state and try to establish a communication session at a later point in time. The unspecified channel recovery duration of telemetry module 22 of external device 18 is typically longer than that of IMD 20.

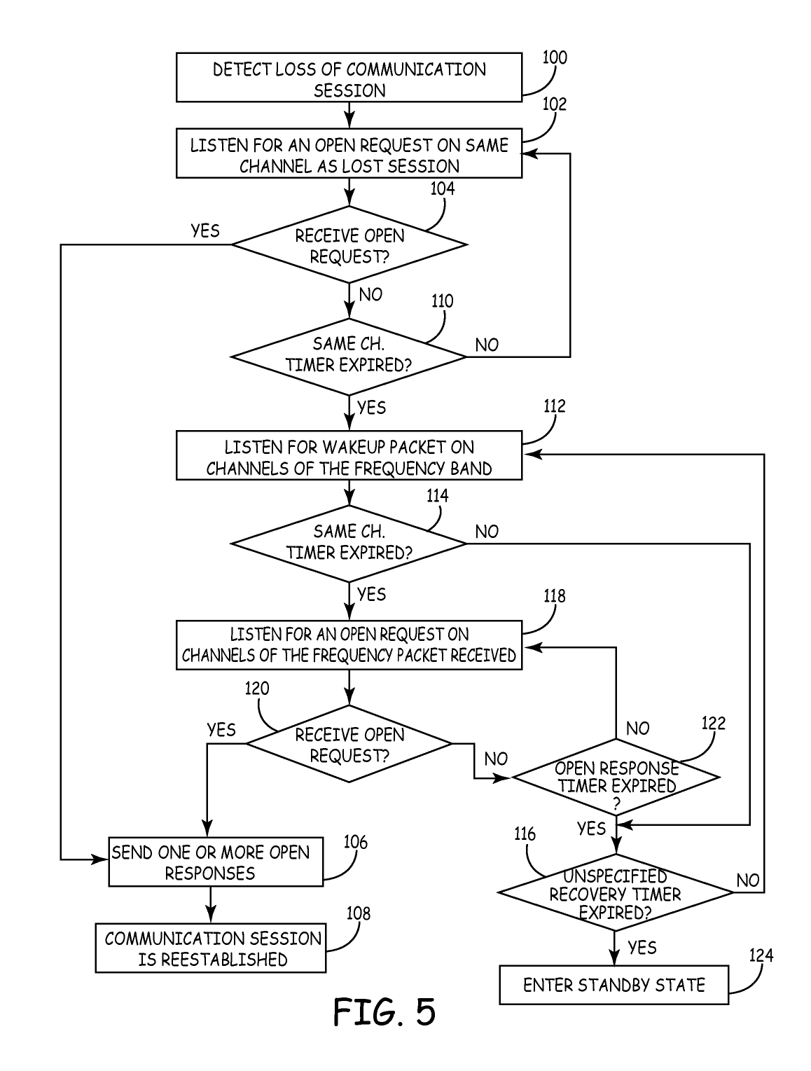

FIG. 5 is a flow diagram illustrating example operation of a telemetry module 38 of an IMD 20 reestablishing a communication session with an external device 18. Telemetry module 38 of IMD 20 detects loss of an established communication session with an external device 18 (100). As described above, telemetry module 38 of an IMD 20 may detect loss of an established communication channel when no valid packet is received from external device 18 for a particular number of consecutive frames, when a valid packet is received but from a device other than external device 18, e.g., an unexpected external device or another IMD, or the like. Telemetry module 38 of IMD 20 listens for an open request on the same channel as the established communication session that was lost (102).

When an open request packet is received from external device 18 (“YES” branch of 104), telemetry module 38 of IMD 20 sends one or more open response packets (106) and the communication channel is reestablished when external device 18 as soon as external device 18 receives one of the open response packets (108). When no open request packet is received (“NO” branch of 104), telemetry module 38 of IMD 20 determines whether a same channel recovery timer has expired (110). When the same channel recovery timer has not expired (“NO” branch of 110), telemetry module 38 of IMD 20 continues to listen for open request packets on the same channel.

When the same channel recovery timer has expired (“YES” branch of 110), telemetry module 38 of IMD 20 aborts the same channel recovery and attempts to reestablish communication with external device 18 using unspecified channel recovery. In the unspecified channel recovery, telemetry module 38 of IMD 20 listens for a wakeup packet on a plurality of channels of a frequency band for a first period of time (112). When no wakeup packet is received during the particular period of time (“NO branch of 114), telemetry module 38 of IMD 20 determines whether an unspecified channel recovery timer has expired (116). When the unspecified channel recovery timer has not expired (“NO” branch of 116), telemetry module 38 of IMD 20 listens for a wakeup packet on the plurality of channels of the frequency band for a second period of time. In this manner, telemetry module 38 of IMD 20 may be viewed as asynchronously sniffing for wakeup packets with little time between sniff intervals.

Upon receiving a wakeup packet during one of the time periods during which telemetry module 38 of IMD 20 listens for wakeup packets (“YES” branch of 114), telemetry module 38 of IMD 20 listens for an open request packet on the channel on which the wakeup packet was received (118). When an open request packet is received from external device 18 (“YES” branch of 120), telemetry module 38 of IMD 20 sends one or more open response packets (106) and the communication channel is reestablished when external device 18 as soon as external device 18 receives one of the open response packets (108).

When no open request packet is received (“NO” branch of 120), telemetry module 38 of IMD 20 determines whether an open request timer has expired (122). When the open request timer has not expired (“NO” branch of 122), telemetry module 38 of IMD 20 listens for an open request packet on the channel on which the wakeup packet was received. When the open request timer has expired (“YES” branch of 122), telemetry module 38 of IMD 20 determines whether an unspecified channel recovery timer has expired (116). When the unspecified channel recovery timer has not expired (“NO” branch of 116), telemetry module 38 of IMD 20 reverts back to listening for wakeup packets.

When the unspecified channel recovery time has expired (“YES” branch of 116), telemetry module 38 of IMD 20 may enter a standby state (124). In other words, if neither the same channel recovery nor the unspecified channel recovery is successful, telemetry module 38 may enter a low power state and try to establish a communication session at a later point in time. For example, telemetry 38 of IMD 20 may begin listening for wakeup packets at a particular sniff interval (e.g., every two seconds). The sniff interval may be set to be a longer or shorter period of time than two seconds and may be on the order of milliseconds, minutes, hours or the like. In the standby state, the amount of power consumed by components of telemetry module 38 are reduced, but the amount of time required to establish a communication session (e.g., latency) is increased as compared to the specified and unspecified channel recovery.

The techniques described above provide faster recovery times of the communication session as compared with a situation in which the IMD scans (listens) on each of the channels in the native communication mode for an open request. Below is a chart comparing the worst case scenarios of unspecified channel recovery using telemetry wakeup as described in this disclosure (first column) vs. the situation in which the IMD scans (listens) on each of the channels in the native communication mode (second column).

| Unspecified channel recovery using | Unspecified channel recovery using |
|---|---|
| telemetry wakeup (worst case) | native mode scanning (worst case) |
| 70 msec (97 Kbps, using 2 wakeup | 1030 msec (97 Kbps, MICS only) |
| packets of~31 msec) | |
| 76 msec (97 Kbps, using 2 wakeup | 1750 msec (48 Kbps, MICS only) |
| packets~31 msec) | |
| 196 msec (MEDS wakeup packets | 3090 msec (97 Kbps, MICS + MEDS) |
| of~93 msec) | |
| 202 msec (MEDS wakeup packets | 5250 msec (97 Kbps, MICS + MEDS) |
| of~93 msec) | |

The techniques described in this disclosure may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, or other devices. The term “processor” or “processing circuitry” may generally refer to any of the foregoing circuitry, alone or in combination with other circuitry, or any other equivalent circuitry.

Such hardware, software, or firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples have been described. Although the channel recovery techniques of this disclosure are described in the context of a two-staged channel recovery (i.e., same channel recovery followed by unspecified channel recovery), the techniques may be used in a single stage channel recovery. For example, channel recovery may be performed using the telemetry wakeup feature immediately after or soon after loss of the communication session is detected. In other words, there may be no same channel recovery performed in the native mode. These and other examples are within the scope of the following claims.

1. A device for reestablishing a communication session comprising:
an antenna; and
a telemetry module coupled to the antenna, the telemetry module configured to detect loss of an established communication session with a telemetry module of a device, attempt to reestablish the communication session on a same channel as the established communication session that was lost, and attempt to reestablish the communication session on any channel of a plurality of available channels using a wakeup feature of the telemetry module upon the failure to reestablish the communication session on the same channel.

2. The device of claim 1, wherein the telemetry module includes a wakeup communication module that listens for a wakeup packet on the plurality of available channels during a first period of time and, in response to not detecting the wakeup packet during the first period of time, listens for the wakeup packet on the plurality of available channels during a second, consecutive period of time.

3. The device of claim 2, wherein the telemetry module includes a native communication module that, upon receiving the wakeup packet during one of the first period of time and the second period of time, listens for a packet indicating a desire to open a communication session during a third period of time on the one of the plurality of available channels on which the wakeup packet was received or on one of the plurality of available channels indicated within the wakeup packet.

4. The device of claim 3,
wherein the wakeup communication module listens for the wakeup packet in a first communication mode; and
wherein the native communication module listens for the packet indicating the desire to open the communication session in a second communication mode.

5. The device of claim 3, wherein the wakeup communication module listens for another wakeup packet on the plurality of available channels for a fourth period of time when no packet indicating the desire to open the communication session is received during the third period of time.

6. The device of claim 3, wherein the native mode communication module receives the packet indicating a desire to open a communication session on the one of the plurality of available channels and sends one or more packets indicating a desire to open the communication session on the one of the plurality of available channels to reestablish the communication session.

7. The device of claim 2, wherein the wakeup communication module listens for the wakeup packet on two or more of the plurality of available channels concurrently.

8. The device of claim 2, wherein the wakeup communication module listens for the wakeup packet on the plurality of available channels using a multi-phase detection scheme and stops listening for the wakeup packet when signal requirements of at least one of the phases is not met.

9. The device of claim 1, wherein the telemetry module includes:
a native communication module that transmits one or more packets indicating a desire to open a communication session on the same channel during same channel recovery; and
a wakeup communication module that, upon failure to reestablish the communication session on the same channel, selects a channel from the plurality of available channels, transmits one or more wakeup packets on the selected channel, and transmits one or more packets indicating a desire to open a communication session on the selected channel subsequent to the transmission of the one or more wakeup packets.

10. A method for reestablishing a communication session comprising:
detecting loss of an established communication session with a telemetry module of a device;
attempting to reestablish the communication session on a same channel as the established communication session that was lost; and
attempting to reestablish the communication session on any channel of a plurality of available channels using a wakeup feature of the telemetry module upon the failure to reestablish the communication session on the same channel.

11. The method of claim 10, wherein attempting to reestablish the communication session on an unspecified channel using a wakeup feature of the telemetry module comprises:
listening for a wakeup packet on the plurality of available channels during a first period of time; and
in response to not detecting the wakeup packet during the first period of time, listening for the wakeup packet on the plurality of available channels during a second, consecutive period of time.

12. The method of claim 11, further comprising:
receiving the wakeup packet on one of the plurality of available channels during one of the first period of time and the second period of time; and
upon receiving the wakeup packet, listening for a packet indicating a desire to open a communication session during a third period of time on the one of the plurality of available channels on which the wakeup packet was received or one of the plurality of available channels indicated within the wakeup packet.

13. The method of claim 12,
wherein listening for the wakeup packet comprises listening for the wakeup packet in a first communication mode; and
wherein listening for the packet indicating the desire to open the communication session comprises listening in a second communication mode.

14. The method of claim 12, further comprising listening for another wakeup packet on the plurality of available channels for a fourth period of time when no packet indicating the desire to open the communication session is received during the third period of time.

15. The method of claim 12, further comprising:
receiving the packet indicating a desire to open a communication session on the one of the plurality of available channels; and
sending one or more packets indicating a desire to open the communication session on the one of the plurality of available channels to reestablish the communication session.

16. The method of claim 11, wherein listening for the wakeup packet comprises listening for the wakeup packet on two or more of the plurality of available channels concurrently.

17. The method of claim 11, wherein listening for the wakeup packet comprises listening for the wakeup packet on the plurality of available channels using a multi-phase detection scheme and stops listening for the wakeup packet when signal requirements of at least one of the phases is not met.

18. The method of claim 10,
wherein attempting to reestablish the communication session on the same channel comprises transmitting one or more packets indicating a desire to open a communication session on the same channel; and
wherein attempting to reestablish the communication session on an unspecified channel using the wakeup feature of the telemetry module comprises:
selecting a channel from the plurality of available channels;
transmitting one or more wakeup packets on the selected channel; and
transmitting one or more packets indicating a desire to open a communication session on the selected channel subsequent to the transmission of the one or more wakeup packets.

19. A device comprising:
means for detecting loss of an established communication session with a telemetry module of a device;
means for attempting to reestablish the communication session on a same channel as the established communication session that was lost; and
means for attempting to reestablish the communication session on any channel of a plurality of available channels using a wakeup feature of the telemetry module upon the failure to reestablish the communication session on the same channel.

20. The device of claim 19, wherein the means for attempting to reestablish the communication session on any channel of a plurality of available channels using a wakeup feature comprises:
means for listening for a wakeup packet on the plurality of available channels during a first period of time and, in response to not detecting the wakeup packet during the first period of time, the means for listening listens for the wakeup packet on the plurality of available channels during a second, consecutive period of time; and
upon receiving the wakeup packet, means for listening for a packet indicating a desire to open a communication session during a third period of time on the one of the plurality of available channels on which the wakeup packet was received or on the one of the plurality of available channels indicated within the wakeup packet.

21. The device of claim 20,
wherein listening for the wakeup packet comprises listening for the wakeup packet in a first communication mode; and
wherein listening for the packet indicating the desire to open the communication session comprises listening in a second communication mode.

22. The device of claim 20, wherein the means for listening for the wakeup packet listens for the wakeup packet on two or more of the plurality of available channels concurrently.

23. The device of claim 20, wherein the means for listening for the wakeup packet listens for the wakeup packet on the plurality of available channels using a multi-phase detection scheme and aborts the listening for a wakeup packet when signal requirements of at least one of the phases is not met.

24. The device of claim 19,
wherein the means for attempting to reestablish the communication session on the same channel transmits one or more packets indicating a desire to open a communication session on the same channel; and; and
means for attempting to reestablish the communication session on any channel of the plurality of available channels using the wakeup feature selects a channel from the plurality of available channels, transmits one or more wakeup packets on the selected channel, and transmits one or more packets indicating a desire to open a communication session on the selected channel subsequent to the transmission of the one or more wakeup packets.
